(19) United States
(12) Patent Application Publication
(10) Pub. No.: US 2013/0226293 A1
(43) Pub. Date: Aug. 29, 2013

(54) ACCOMMODATIVE IOL - REFRACTIVE INDEX CHANGE THROUGH CHANGE IN POLARIZABILITY OF A MEDIUM

(71) Applicant: Novartis AG, (US)

(72) Inventor: Krishnakumar Venkateswaran, Burleson, TX (US)

(73) Assignee: Novartis AG, Fort Worth, TX (US)

(21) Appl. No.: 13/775,517

(22) Filed: Feb. 25, 2013

Related U.S. Application Data
(60) Provisional application No. 61/602,281, filed on Feb. 23, 2012.

(51) Int. Cl. A61F 2/16

(52) U.S. Cl. CPC A61F 2/1627
USPC 623/6.22; 623/6.37

(57) ABSTRACT

In one aspect, an accommodative intraocular lens (IOL) is disclosed that includes an optic having at least a portion formed of a polarizable and/or and electro-active material. Once implanted in a subject's eye, a change in the index of refraction of the polarizable and/or electro-active portion in response to forces applied to the optic via the eye's ciliary muscle can cause a change in the optical power of the optic, thereby allowing accommodation.

### RELATED APPLICATIONS

This application claims priority to U.S. provisional application Ser. No. 61/602,281, filed on Feb. 23, 2012, the contents which are incorporated herein by reference.

### TECHNICAL FIELD

This invention relates generally to the field of intraocular lenses (IOLs) and, more particularly, to accommodative IOLs.

### BACKGROUND OF THE INVENTION

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and ultrasonically vibrated. The vibrating cutting tip liquifies or emulsifies the lens so that the lens may be aspirated out of the eye. The diseased lens, once removed, is replaced by an artificial lens.

In the natural lens, bifocality of distance and near vision is provided by a mechanism known as accommodation. The natural lens is contained within the capsular bag and is soft early in life. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In this way, the natural lens can focus alternatively on near and far objects.

As the lens ages, it becomes harder and is less able to change its shape in reaction to the tightening of the ciliary muscle. This makes it harder for the lens to focus on near objects, a medical condition known as presbyopia. Presbyopia affects nearly all adults over the age of 45 or 50. Accordingly, there exists a need for better solutions to the problem of accommodation in IOLs.

### SUMMARY

In one aspect, an accommodative intraocular lens (IOL) is disclosed, which includes an optic adapted for implantation in the human eye, where the optic includes at least a portion formed of an electro-active material and a transducer in electrical communication with the electro-active material for application of an electric field thereto. The transducer is mechanically coupled with the ciliary muscle when the lens is implanted in the eye such that the transducer can modulate the electric field it applies to the electro-active material in response to ciliary muscle movements to adjust the refractive index of the electro-active material so as to facilitate accommodation.

A variety of electro-active materials can be used in the above IOL. While in some embodiments the electro-active material comprises a liquid crystal, in others it can be a polymeric material. Some examples of suitable electro-active liquid crystals include, without limitation, nematic liquid crystals, such as pentyl-cyano-biphenyl, (n-octyloxy)-4-cyanobiphenyl. Other examples of liquid crystals can include 4-cyano-4-n-alkylbiphenyl, 4-n-pentyloxy-biphenyl, 4-cyano-4″-n-alkyl-p-terphyls, where n=3, 4, 5, 6, 7, 8 or 9. Some examples of suitable polymeric electro-active materials include polymers, such as polystyrene, polycarbonate, polymethylmethacrylate, polyvinylcarbazole, polyimide, polysilane, containing chromophores, such as paranitroaniline (PNA), disperse red 1 (DR 1), 3-methyl-4-methoxy-4′-nitrostilbene, diethylaminonitrostilbene (DANS), and diethyl-thio-barbituric acid.

The optic of the above IOL can include an anterior surface and a posterior surface with a variety of different profiles, e.g., convex-convex, convex-concave, convex-flat, concave-flat, among others. In some embodiments, at least one of said anterior or posterior surfaces of the optic is formed of the electro-active material. Alternatively, the entire optic can be formed of the electro-active material.

In some embodiments, the optic can include a core portion, e.g., formed of a biocompatible material, and the electro-active material can be disposed as a layer on at least a surface of the core portion. For example, the core portion can include an anterior surface and a posterior surface and the electro-active material can be disposed as a layer on at least a portion of those surfaces.

The core portion can be formed of one or more biocompatible polymers. Some examples of suitable polymers include, without limitation, any of a soft acrylic, hydrogel and silicone. For example, the biocompatible polymer can include polymethylmethacrylate and a copolymer of 2-phenylethylacrylate/2-phenylethyl methacrylate.

In some embodiments, in the above IOL, the optic includes a core portion in the form of a flexible shell having an anterior surface and a posterior surface, where the shell is adapted to be in mechanical coupling with the ciliary muscle when the lens is implanted in the eye such that ciliary muscle movements alter the curvature of at least one of the anterior or posterior surfaces so as to facilitate accommodation. The electro-active material can be housed within the shell so as to be in electrical communication with the transducer.

The IOL can include a pair of haptics for fixating the optic in the eye and providing a mechanism for transmitting compressive and/or tensile forces from the ciliary muscle to the transducer.

In another aspect, an accommodative IOL is disclosed, which includes an optic adapted for implantation in the human eye, where the optic includes at least a polarizable portion that exhibits a change in its refractive index in response to a change in pressure applied thereto. The polarizable portion is adapted to be in mechanical coupling with the ciliary muscle when the lens is implanted in the eye such that the movements of the ciliary muscle can modulate pressure applied to the polarizable portion, thereby changing its refractive index and adjusting an overall power of the optic for facilitating accommodation.

The accommodative IOL can include one or more haptics that are mechanically coupled to the polarizable portion for fixating the optic in the eye. The haptics are adapted for coupling with the ciliary muscle when the optic is implanted in the eye so as to facilitate application of pressure to said polarizable portion in response to the movements of the ciliary muscle.

In some embodiments, the above accommodative IOL can further include a pressure amplifier coupled to said haptics for amplifying pressure applied to said haptics in response to the movements of the ciliary muscle.

In some embodiments, the polarizable portion exhibits a change in a range of about 10% to about 25%, or in some instances at least about 16%, in its refractive index in response to a change in pressure in a range of about 35 MPa applied thereto, e.g., by changing the applied pressure from about 5 MPa to about 50 MPa.

In some embodiments, the optic comprises a shell for housing the polarizable portion. In some cases, the shell is formed of a flexible material and includes an anterior surface and a posterior surface such that when the optic is implanted in the eye the ciliary muscle movements alter the curvature of at least one of the anterior and posterior surfaces so as to facilitate accommodation, e.g., by augmenting the effect of the polarizable portion on the optical power of the optic.

In many embodiments, the shell is formed of a biocompatible material, such as soft acrylic, hydrogel and silicone. By way of example, the shell can be formed of a cross-linked copolymer of 2-phenylethyl acrylate and 2-phenylethyl methacrylate.

In another aspect, an accommodative intraocular lens is disclosed, which includes a plurality of optics that are adapted for implantation in a subject's eye, where the optics collectively provide the subject with an optical power. Each of the optics has at least a polarizable and/or an electro-active portion that is adapted to be mechanically coupled with the ciliary muscle when the optics are implanted in the eye, where the polarizable portion exhibits a change in its refractive index in response to a change in pressure applied thereto and the electro-active portion exhibits a change in its index of refraction in response to a change of electric field applied thereto (e.g., generated via a voltage change), in some embodiments in which the optics include polarizable portions, the movements of the ciliary muscle modulate pressure applied to those polarizable portions, thereby adjusting an overall power of the lens for facilitating accommodation. In some embodiments in which the optics include electro-active portions, the movements of the ciliary muscle can modulate pressure applied to a transducer, which in turn modulate the voltage applied to the electro-active portions, thereby adjusting an overall power of the lens. In some embodiments, the optics can include both polarizable and electro-active portions. Further in some embodiments, a polarizable portion can be formed of a material that also functions as an electro-active material.

In some embodiments, in the above accommodative IOL, the plurality of optics 22 collectively provide an accommodative power (i.e., an add power for near vision) in a range of about 3 D to about 4 D. In some embodiments, each optic provides an accommodative power in a range of about 0.2 D to about 2 D, e.g., in a range of about 1 D to about 2 D. The number of optics can vary from one embodiment to another, but typically is in a range of about 5-20, e.g., 5-10. In some embodiments, the thickness of each optic can be selected, e.g., based on the radius of curvatures of its surfaces, the electrical and mechanical properties of the material(s) forming the optics, e.g., a biocompatible polymer such as those disclosed herein, as well as the particular application for which the IOL is intended.

In some embodiments, each optic can have a thickness in a range of about a few hundred nanometers to about 1 micrometer (micron). In some embodiments, the above accommodative IOL further includes a coupling mechanism, e.g., a ring, for mechanically coupling the optics to one another. Further, the IOL can include one or more haptics for fixating the optics in the eye. By way of example, the haptics can be attached, integrally or otherwise, to the coupling mechanism that holds the optics together.

In some embodiments, the above accommodative IOL further includes a pressure amplifier coupled to the polarizable portions of the optics and/or a pressure transducer coupled to the electro-active portions of the optics, which are adapted for coupling with the ciliary muscle when the optics are implanted in the eye to facilitate application of pressure and/or voltage to the polarizable and/or electro-active portions in response to movements of the ciliary muscle.

In many embodiments, the optics are formed of biocompatible materials, such as those discussed above.

Further understanding of the invention can be obtained by reference to the following detailed description in conjunction with the attached drawings, which are described briefly below.

### BRIEF DESCRIPTION OF DRAWINGS

FIG. 1A schematically depicts an IOL according to an embodiment of the invention,

FIG. 1B schematically depicts an IOL according to another embodiment of the invention,

FIG. 2A schematically depicts an IOL according to another embodiment of the invention that includes a plurality of thin optics each of which has at least a portion formed of a polarizable material,

FIG. 2B schematically depicts one of the thin optics of the IOL of FIG. 2A,

FIG. 3 schematically depicts an IOL according to another embodiment of the invention that employs an electro-active material for changing the optical power of the JUL in response to forces applied to the IOL via ciliary muscle during accommodation, and

FIG. 4 schematically depicts an IOL according to another embodiment having an optic on at least one of surface of which an electro-active layer is disposed.

### DETAILED DESCRIPTION

The present invention generally provides accommodative lenses, and in particular accommodative intraocular lenses (IOLs), that utilize polarizable and/or electro-active materials to change the index of refraction of one or more optics of the lenses in response to forces exerted on the optics via contraction and relaxation of the eye's ciliary muscle as the subject attempts to view near and far objects. The embodiments discussed below are exemplary, and various changes can be made to these illustrative embodiments without deviating from the scope of the invention. For example, the features of one embodiment can be combined with those of another embodiment.

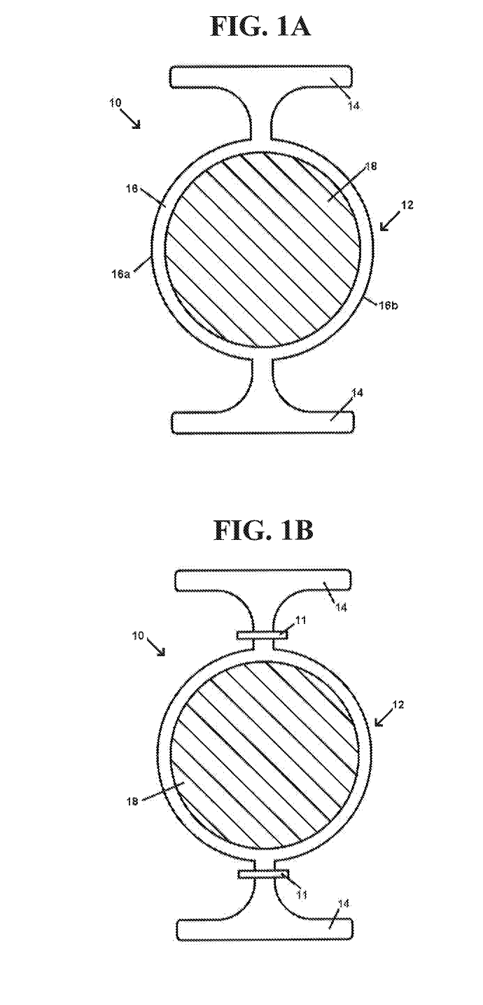

FIG. 1A schematically depicts an intraocular lens (IOL) 10 according to one embodiment of the invention that includes an optic 12 for providing an accommodating optical power and a pair of opposing haptics 14 for placement of the lens within the capsular bag of a patient's eye. In this implementation, the haptics 14 are generally T-shaped and are configured to be in mechanical communication with the ciliary muscle via the capsular bag. The haptics 14 are configured to stretch and fill the equatorial region of the capsular bag when the lens is implanted in the eye.

The optic 12 includes a hollow shell 16 that houses a polarizable material 18. The shell 16 includes an anterior surface 16a and a posterior surface 16b. The term “polarizable material” as used herein refers to a material whose index of refraction for at least one wavelength of visible light changes in response to a change in applied pressure, e.g., the refractive index of the polarizable material can exhibit a change of at least about 16% in response to a change of about 35 MPa (mega pascals) in the applied pressure, e.g., a change of pressure from about 5 MPa to about 40 MPa, which can be applied in some embodiments via a piezoelectric pressure transducer to the polarizable material. The polarizable material can be a solid, a liquid or a gas. Some examples of suitable polarizable materials include, without limitation, electro-active polymers such as polyvinylidene fluoride (PVDF). In some embodiments, the polarizable material can be a conductive polymer, such as polypyrrole and polyaniline. In some embodiments, the low operating voltages of such conductive polymers make their use as the polarizable material attractive.

As shown schematically in FIG. 1B, in some embodiments, the lens 10 can further include a pair of pressure amplifiers 11 each of which is positioned between a respective haptic and the optic 12. The pressure amplifiers can transmit and amplify the pressure applied to the haptics by the ciliary muscle to the polarizable material 18. In some embodiments, the pressure amplifiers can include micro-electronic circuits for amplifying pressure applied to the polarizable material. In some embodiments, the amplifiers can include charge amplifiers for amplifying small changes in electric charge density due to changes in applied pressure.

The shell 16 can be formed of a variety of suitable biocompatible materials, such as biocompatible polymers. Some examples of such suitable biocompatible polymers include, without limitation, soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®.

In embodiments in which the polarizable material is a liquid, the shell provides a sealed enclosure for housing the liquid so as to inhibit its leakage into the eye.

The IOL 10 can be implanted in a subject's capsular bag such that the haptics 14 receive mechanical force from the eye's ciliary muscle. As the subject attempts to view far objects, e.g., objects at a distance greater than about 10 cm, the ciliary muscle relaxes, thereby reducing the force exerted on the haptics. This in turn reduces the pressure exerted on the polarizable material 18 housed within the shell 16. In contrast, as the subject attempts to view near objects, the ciliary muscle tightens up, thereby increasing the force exerted on the haptics 14. This in turn increases the pressure exerted on the polarizable material.

The curvature of the anterior and posterior surfaces of the shell, the index of refraction of the material forming the shell as well as the index of refraction of the polarizable material can be selected so that the IOL would. exhibit a desired far-focus optical power (that is, a desired optical power when the ciliary muscle is relaxed). By way of example, in this exemplary embodiment, the IOL 10 provides a far-focus optical power of about 34 D. As the subject tries to focus on closer objects, the pressure exerted on the polarizable material causes a change in the index of refraction of the material, thereby changing (increasing) the optical power of the lens. For example, in some embodiments, the maximum pressure applied by the ciliary muscle on the optic for near vision accommodation can lead to an increase of the lens's optical power by a value in a range of about 3 Diopters to about 4 Diopters.

Without being limited to any particular theory, the change in the index of refraction of the polarizable material and consequently that of the lens can be understood by considering that the electronic contribution to the index of refraction of the polarizable material can be proportional to the charge density of the material. Hence, an increase in the pressure applied to the polarizable material can cause an increase in the charge density of the material, thereby increasing its index of refraction. Again, without being limited to any particular theory and by way of further explanation, the index of refraction (η) of the polarizable material can satisfy the following equation (commonly known as the Claussius Mossoti equation):

$$3 \frac{{n}^{2} - 1}{{n}^{2} + 2} = N α , \qquad Eq . ( 1 )$$

wherein,

η represents the index of refraction of the material,

α represents the atomic polarizability of the material, and

N represents the number of molecules per unit volume.

By way of illustration, for water, Nα in the above Eq. (1) can be estimated as 0.617, which implies an index of refraction (η) of 1,3329. Thus, for a 10 D increase in optical power, the value of Nα needs to be changed to 0.7795, which results in a refractive index of 1.4329. Since the atomic polarizability (α) is proportional to the square of charge density, this exemplary calculation suggests that a 12% change in charge density of water, for example, can cause a 10 D change in optical power. In some embodiments, the polarizable material can include, without limitation, dielectric elastomers and electrorheological fluids, which exhibit a reversible change in viscosity in response to application of an electric field. The electric and mechanical properties of the dielectric polymers will change with pressure, thereby causing a change in their optical properties.

In some embodiments, pressure points having small areas are employed to increase force transfer. In the case of ciliary muscles, the force exerted on the capsular bag can be of the order of a few milli-Newtons (mN). To generate a pressure of a few MPa, in some embodiments, the contact area of the force transfer can be of the order of one square millimeter (a force of 1 mN over an area of 1 square millimeter will translate into a pressure of 1 KPa). In some embodiments, the electric charge generated in the polarizable material can be amplified using charge amplifiers.

Referring again to FIGS. 1A and 1B, in some embodiments, the shell 16 can be flexible to allow a change in the curvature of at least one of, and preferably both of, its anterior and posterior surfaces in response to a change in pressure applied by the haptics to the optic 12. The change in the curvature of these surfaces can in turn facilitate accommodation by augmenting the effect of the change in the index of refraction of the polarizable material on the optical power of the lens. By way of example, a compressive pressure on the optic 12 can cause the anterior surface to vault in an anterior direction and the posterior surface to vault in a posterior direction, thereby increasing the radius of curvature of the surface and hence the optical power of the lens. This increase in the optical power of the lens due to a change in the curvature of these surfaces can in turn augment the change in the optical power due to a change in the index of refraction of the polarizable material so as to provide an overall desired near focus power.

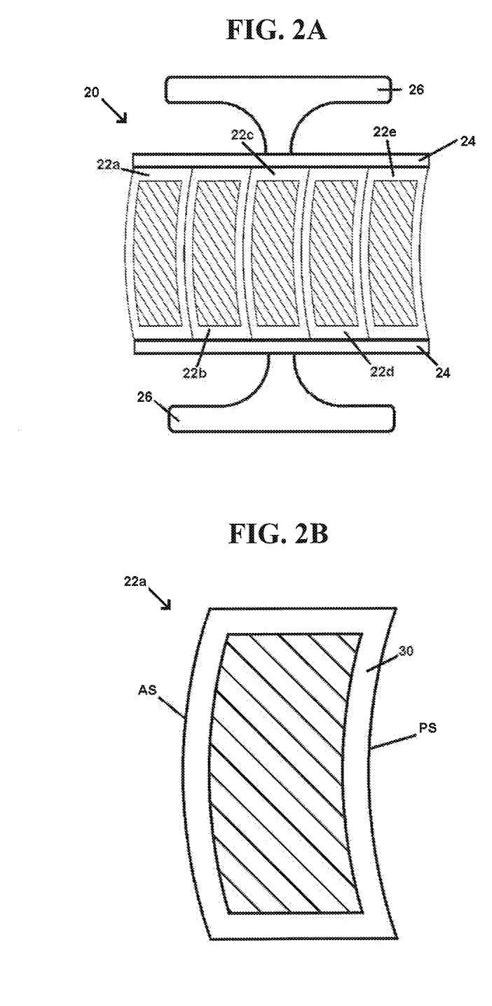

In some embodiments, a lens according to the teachings of the invention can include a plurality of thin optics that collectively provide optical power to the patient, where each of the optics includes a polarizable material that can facilitate accommodation via a change in its index of refraction in response to pressure applied by the ciliary muscle. By way of example, FIG. 2A schematically depicts an accommodative intraocular lens 20 according to such an embodiment of the invention that includes a plurality of optics 22a, 22b, 22c, 22d, and 22e (herein collectively referred to as optics 22) that are coupled to a ring 24 for placement in a patient's eye, e.g., in the capsular bag. The lens 20 further includes a pair of T-shaped haptics 26 that allow mechanical coupling of the optics 22, via the ring 24, with the ciliary muscle to receive tensile or compressive forces from the ciliary muscle during accommodation. In this implementation, the number of optics is 5, but in other implementations, the number of optics can be different. For example, the number of optics can be in a range of 2 to 20, or 5 to 10 depending on the radius of curvature of the optics, their thickness, and characteristics of a housing in which the optics are disposed.

Each of the optics includes at least a portion formed of a polarizable material that is transparent, or at least substantially transparent, to visible radiation. In some cases, the entire optic can be formed of a polarizable material. In other cases, one or more of the optics can be formed as a hollow shell within which a polarizable material is disposed. By way of example, FIG. 2B schematically shows one of the optics 22 (e.g., optic 22a) that includes a polarizable material, e.g., water, which is encased in a polymeric shell 30 formed of a biocompatible polymer such as those listed above. The polymeric shell includes a convex anterior surface (AS) and a concave posterior surface (PS), although other shapes such as convex-convex, convex-concave, or concave-concave, or flat-flat can also be utilized. In this embodiment, each of the optics 22 has a thickness (t), e.g., in a range of about few hundred nanometers to about 1 micron with the maximum thickness of the optics being constrained by the size of the capsular bag.

Once implanted in the eye, the haptics 26 engage with the ciliary muscle to transmit compressive or tensile pressure to the optics via the ring 24 as the patient attempts to accommodate from far to near or near to far. The optics 22 are configured such that, once implanted in the eye, they collectively provide a desired optical power for far vision when the eye's ciliary muscle is relaxed and the force on the haptics is low. For example, the collective optical power provided by the optics 22 can be about 34 D. As known by those of ordinary skill in the art, the indices of refraction of the constituents of the optics (e.g., polymeric casing and the polarizable material), the index of refraction of the medium surrounding the optics when the lens is implanted in the eye (e.g., the index of refraction of the aqueous humour) as well as the profiles of the anterior and posterior surfaces of the optics can be configured to obtain a desired far-vision optical power.

As the subject tries to view near objects, the pressure on the polarizable portions of the optics 22 due to the force exerted on the haptics as a result of contraction of the ciliary muscle can cause a change in the index of refraction of those portions in a manner discussed above and hence change the optical power of the lens 20. The small size of each individual optic facilitates generation of sufficient pressure on the polarizable material in response to force exerted on the haptics due to contraction of the ciliary muscle that would yield a desired change in the index of refraction of the polarizable material, and hence the optical power of the optic. In some embodiments, the optics 22 can cooperatively provide a change in the index of refraction of the IOL that would lead to an accommodative power in a range of about 3 D to about 4 D.

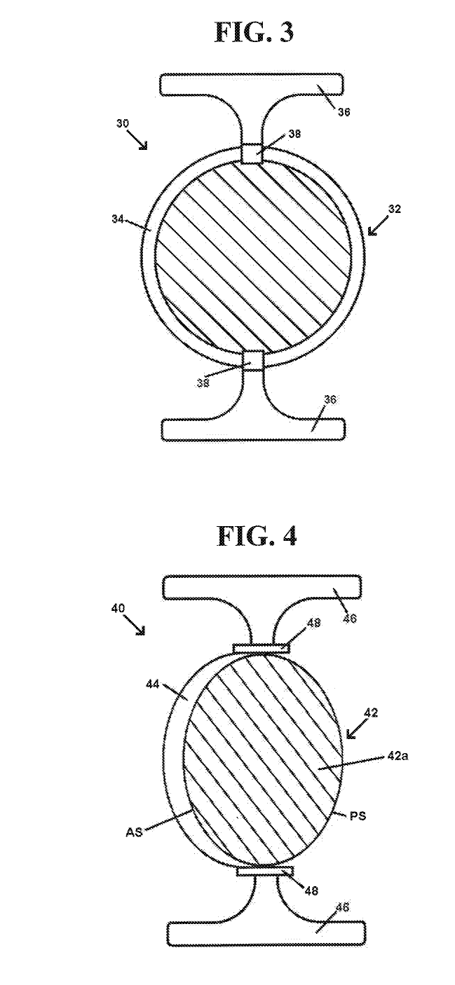

In some embodiments, in addition to or instead of a polarizable material, an electro-active material, which exhibits a change in its index of refraction in response to an applied electric field, can be employed. In such embodiments, the IOL can include a pressure transducer, e.g., a piezoelectric element, that can convert the pressure applied by the ciliary muscle on the haptics into an electric field for application to the polarizable material. By way of example, FIG. 3 schematically depicts an intraocular lens 30 according to such an embodiment that includes a shell 32 for housing an electro-active material 34 and a pair of T-shaped haptics 36. In addition, the intraocular lens 30 includes a pressure transducer 38 that is adapted to receive compressive or tensile forces from the haptics and is in electrical communication with the electro-active material 34 (e.g., via a pair of electrodes). The pressure transducer, which can include, e.g., a piezoelectric element, can convert the applied force into an electric voltage for application across the electro-active material.

For example, as the patient attempts to view near objects, the ciliary muscle contracts, thus resulting in the application of a compressive force on the haptics 36, which in turn transmit this force to pressure transducer 38. The pressure transducer then applies an electric voltage (and a concomitant electric field) to the electro-active material 34 to change its index of refraction. For example, the applied electric field can cause an increase in the index of refraction of the electro-active material and hence the optical power of the lens so as to provide accommodation. Some examples of suitable electro-active materials include, e.g., liquid crystals and electro-active polymeric materials discussed further below.

The teachings of the invention can be implemented in a variety of ways, and are not limited to the embodiments described above. In the above IOL 20, the optics 22 can include, in addition to or instead of polarizable portions, electro-active portions whose indices of refraction change in response to application of a voltage thereto. For example, one or more transducers can be provided, e.g., in a manner depicted in the above IOL 30, to apply voltage to the electro-active portions in response to movements of the ciliary muscle.

In some embodiments, an IOL according to the teachings of the invention can include a layer of an electro-active material disposed on an anterior or a posterior surface of its one or more optic(s) to provide an accommodative change in the optical power of the IOL. By way of example, FIG. 4 schematically depicts an IOL 40 according to such an embodiment of the invention that includes an optic 42 formed of a biocompatible material, such as those discussed above. In this embodiment the optic 42 includes a core portion 42a having a convex-convex profile, though other profiles such as convex-fiat, convex-concave, or concave-concave can also be employed. The core portion 42a includes an anterior surface (AS) and a posterior surface (PS).

The IOL 40 further includes a layer of an electro-active material 44 that is disposed on the anterior surface (AS) of the core portion 42a. The electro-active material is transparent to visible optical radiation. In some embodiments, the electro-active layer 44 can have a thickness in a range of about a few hundred nanometers to a few microns, e.g., depending on whether a polymer forming the electro-active material is single layer or composite. The IOL 40 further includes a pair of haptics 46 that facilitate its placement in a patient's eye and its mechanical engagement with the eye's ciliary muscle, and a pair of pressure transducer 48 that are in mechanical coupling with the haptics 46 and electrical coupling with the electro-active layer 44. The tensile or compressive forces exerted by the ciliary muscle on the haptics 46 are transmitted to the pressure transducer, which can in turn generate and apply a voltage across the electro-active layer. The applied voltage causes a change in the index of refraction of the electro-active layer, and hence a change in the optical power of the optic 42. For example, as the subject tries to view near objects, a compressive pressure on the transducers 48 can result in an increase in the index of refraction of the electro-active layer to allow accommodation. For example, the increase in the index of refraction of the electro-active layer can result in an add power in a range of about 3 to about 4 D.

In some embodiments, the electro-active layer can include a liquid crystal, In other embodiments, the electro-active layer can include a polymer gel. Some examples of suitable liquid crystals can include, without limitation, sematic liquid crystals, such as pentyl-cyano-biphenyl, (n-octyloxy)-4-cyanobiphenyl. Other examples of liquid crystals can include 4-cyano-4-n-alkylbiphenyl, 4-n-pentyloxy-biphenyl, 4-cyano-4″-n-alkyl-p-terphyls, where n=3, 4, 5, 6, 7, 8 or 9. Some examples of suitable polymeric electro active materials include polymers, such as polystyrene, polycarbonate, polymethylmethacrylate, polyvinylcarbazole, polyimide, polysilane, containing chromophores, such as paranitroaniline (PNA), disperse red 1 (DR 1), 3-methyl-4-methoxy-4′-nitrostilbene, diethylaminonitrostilbene (DANS), and diethyl-thio-barbituric acid. Further information regarding suitable electro-active materials can be found in U.S. Published Patent Application No. 2004/0051846 entitled “System, Apparatus, And Method For Correcting Vision Using An Electro-Active Lens,” which is herein incorporated by reference in its entirety.

In some embodiments, the optic 42 of the IOL 40 is formed of a flexible material that allows some deformation of the optic in response to pressure from the ciliary muscle to augment the accommodative effect of the electro-active layer. For example, in response to a compressive pressure exerted by the haptics 46, the anterior surface 42a of the optic 42 can vault in an anterior direction and the posterior surface 42b of the optic 42 can vault in a posterior direction. This causes a change in the radius of curvature of these surfaces and hence changes, e.g., increases, the optical power of the lens.

Those having ordinary skill in the art will appreciate that various changes can be made to the above embodiments without departing from the scope of the invention.

What is claimed is:

1. An accommodative intraocular lens, comprising:
an optic adapted for implantation in the human eye, said optic comprising at least a portion formed of an electro-active material, and
a transducer in electrical communication with the electro-active material for application of an electric field thereto,
wherein said transducer is configured for mechanical coupling with the ciliary muscle when the lens is implanted in the eye such that the transducer can modulate the electric field it applies to the electro-active material in response to ciliary muscle movements to adjust refractive index of the electro-active material so as to facilitate accommodation.

2. The lens of claim 1, wherein said electro-active material comprises a liquid crystal.

3. The lens of claim 1, wherein said electro-active material comprises a polymeric material.

4. The lens of claim 1, wherein said optic comprises an anterior and a posterior surface.

5. The lens of claim 4, wherein at least one of said anterior and posterior surfaces is formed at least partially of said electro-active material.

6. The lens of claim 1, wherein said optic comprises a core portion and said electro-active material forms a layer disposed on a surface of said core portion.

7. The lens of claim 3, wherein said core portion is formed of one or more biocompatible polymers.

8. The lens of claim 6, wherein said core portion comprises a flexible shell having an anterior surface and a posterior surface, said shell being in mechanical coupling with the ciliary muscle when the lens is implanted in the eye such that ciliary muscle movements alter the curvature of at least one of said anterior and posterior surfaces so as to facilitate accommodation.

9. The lens of claim 7, wherein said biocompatible material comprises any of a soft acrylic, hydrogel and silicone.

10. The lens of claim 9, wherein said biocompatible material comprises any of polymethylmethacrylate and a copolymer of 2-phenylethylacrylate/2-phenylethyl methacrylate.

11. The lens of claim 1, wherein said transducer comprises one or more piezoelectric elements.

12. The lens of claim 8, wherein said electro-active material is housed in said shell.

13. The lens of claim 1, further comprising one or more haptics for fixating the optic within the eye.

14. An accommodative intraocular lens, comprising
an optic adapted for implantation in the human eye,
said optic having at least a polarizable portion exhibiting a change in its refractive index in response to a change in pressure applied thereto, said portion being configured for mechanical coupling with the ciliary muscle when the lens is implanted in the eye,
wherein movements of the ciliary muscle modulate pressure applied to said polarizable portion, thereby adjusting an overall power of the optic for facilitating accommodation.

15. The lens of claim 14, wherein said optic further comprises haptics mechanically coupled to said polarizable portion and adapted for coupling with the ciliary muscle when the optic is implanted in the eye so as to facilitate application of pressure to said polarizable portion in response to movements of the ciliary muscle.

16. The lens of claim 14, further comprising a pressure amplifier coupled to said haptics for amplifying pressure applied to said haptics in response to movement of the ciliary muscle.

17. The lens of claim 14, wherein said polarizable portion exhibits a change of at least about 10% in its refractive index in response to a change in pressure applied thereto.

18. The lens of claim 14, wherein said optic comprises a shell housing said polarizable portion.

19. The lens of claim 14, wherein said polarizable portion comprises any of single or composite electro-active polymers and electrorheological fluids.

20. The lens of claim 14, further comprising one or more haptics for fixating said optic in the eye such that said polarizable portion is in mechanical coupling with the ciliary muscle.

21. The lens of claim 18, wherein said shell is flexible and includes an anterior surface and a posterior surface such that when the optic is implanted in the eye the ciliary muscle movements alter the curvature of at least one of the anterior and the posterior surfaces so as to facilitate accommodation.

22. The lens of claim 18, wherein said shell is formed of a biocompatible material.

23. The lens of claim 22, wherein said biocompatible material comprises any of a soft acrylic, hydrogel and silicone.

24. An accommodative lens, comprising
a plurality of optics adapted for implantation in a subject's eye, said optics collectively providing the subject with an optical power,
each of said optics having at least a polarizable portion adapted to be in mechanical coupling with the ciliary muscle when said optics are implanted in the eye, said polarizable portion exhibiting a change in its refractive index in response to application of pressure thereto,
wherein movements of the ciliary muscle modulate pressure applied to said polarizable portions of the optics, thereby adjusting an overall power of the lens for facilitating accommodation.

25. The accommodative lens of claim 24, wherein each of said optics provides an accommodative power in a range of about 1 Diopter to about 2 Diopters.

26. The accommodative lens of claim 24, wherein a number of said optics is in a range of about 2 to about 20.

27. The accommodative lens of claim 24, wherein a thickness of each of said optics is in a range of about a few hundred nanometers to about one micron.

28. The accommodative lens of claim 24, further comprising a coupling mechanism for mechanically coupling said optics to one another.

29. The accommodative lens of claim 28, further comprising one or more haptics for fixating the optics in the eye.

30. The accommodative lens of claim 24, further comprising a pressure transducer coupled to said polarizable portions of the optics and adapted for coupling with the ciliary muscle when the optics are implanted in the eye to facilitate application of pressure to the polarizable portions in response to movements of the ciliary muscle.

31. The accommodative lens of claim 24, wherein said optics are formed of a biocompatible material.
